(19) United States
(12) Patent Application Publication
(10) Pub. No.: US 2009/0099050 A1
(43) Pub. Date: Apr. 16, 2009

(54) FACIAL CLEANSING POWDER COMPOSITION

(75) Inventor: Teruyuki NANBU, Hakata-ku (JP)

Correspondence Address: OBLON, SPIVAK, MCCLELLAND MAIER & NEUSTADT, P.C., 1940 DUKE STREET, ALEXANDRIA, VA, 22314, US

(73) Assignee: ASKA CORPORATION Co., Ltd., Hakata-ku (JP)

(21) Appl. No.: 12/052,358

(22) Filed: Mar. 20, 2008

(30) Foreign Application Priority Data
Oct. 10, 2007 (JP) 2007-263908

(51) Int. Cl. A61K 8/55

(52) U.S. Cl. 510/137

(57) ABSTRACT

The invention provides a facial cleansing powder composition exhibiting good latherability even when used with hard water, the composition containing, as a substitute for edetic acid, an environmentally acceptable metal-sequestering agent which is readily incorporated into facial cleansing powder. The facial cleansing powder composition contains a facial cleansing powder base, phytic acid, and starch powder, wherein the phytic acid is supported on the starch powder. The amount of phytic acid is preferably 0.01 to 2.0 wt. %. Preferably, phytic acid is supported on starch powder by mixing an aqueous phytic acid solution with starch powder and drying the mixture.

### BACKGROUND OF THE INVENTION

1. Field of the Invention

The present invention relates to a facial cleansing powder composition, and more particularly to a facial cleansing powder composition exhibiting good latherability even when used with hard water.

2. Background Art

Facial cleansing powder is formed from soap base or fatty acid soap serving as a base.

In general, when soap, including facial cleansing powder, is used for washing the face with hard water, the soap poses a problem in that a soap component is bonded to metal ions (e.g., magnesium ions, iron ions, and calcium ions) contained in hard water, and so-called soap scum is generated, resulting in difficulty in lathering.

In order to solve the problem, attempts have been made to form facial cleansing powder by incorporating a metal-sequestering agent into a soap base so that metal ions are trapped through chelating and that lathering occurs even with hard water.

Tetrasodium edetate (tetrasodium ethylenediaminetetraacetate (EDTA 4Na)), which is a conventionally employed typical metal-sequestering agent, dissolves only slightly in water, and melts only at high temperature.

Hitherto, due to the aforementioned properties, sodium edetate has been incorporated only into shampoos, which have a high water content, or bar soap (see, for example, Japanese Patent Application Laid-Open (kokai) No. 2005-53842). As far as is known to the present applicant, facial cleansing powder containing edetic acid has not yet been provided. Since edetic acid is not degraded by microorganisms, use of edetic acid has begun to come under regulatory control in Europe.

### SUMMARY OF THE INVENTION

In view of the foregoing, an object of the present invention is to provide a facial cleansing powder composition exhibiting good latherability even when used with hard water, the composition containing, as a substitute for edetic acid, an environmentally acceptable metal-sequestering agent which is readily incorporated into facial cleansing powder.

The present inventors conducted extensive studies in search of an environmentally acceptable metal-sequestering agent (as a substitute for edetic acid) which can be readily incorporated into facial cleansing powder, and focused on phytic acid. Phytic acid exhibits a metal-sequestering effect, and notwithstanding its form of viscous liquid, it is easy to handle due to its high water solubility. Moreover, phytic acid is a natural component derived from rice bran. Thus, the present inventors made attempts to incorporate phytic acid into facial cleansing powder. However, they encountered a problem; when an aqueous phytic acid solution is added to a facial cleansing powder base, the base is melted. Therefore, the inventors continued research to solve this problem and found that, when phytic acid is supported on starch powder in advance, and the phytic-acid-supporting starch powder is incorporated into facial cleansing powder, this problem can be solved. The present invention has been accomplished on the basis of this finding.

Accordingly, the present invention provides a facial cleansing powder composition containing a facial cleansing powder base, phytic acid, and starch powder, wherein phytic acid is supported on the starch powder.

The facial cleansing powder composition of the present invention exhibits good latherability even when used with hard water. In addition, the composition causes no environmental problems, since phytic acid incorporated into a facial cleansing powder base is a natural component derived from rice bran.

### DETAILED DESCRIPTION OF PREFERRED EMBODIMENTS

The present invention will next be described in more detail.

The facial cleansing powder composition of the present invention contains a facial cleansing powder base, phytic acid, and starch powder, wherein phytic acid is supported on the starch powder.

No particular limitation is imposed on the facial cleansing powder base employed in the present invention, so long as it is generally employed as a base for facial cleansing powder. Examples of the facial cleansing powder base which may be employed include soap base (obtained through saponification or neutralization); and fatty acid soaps such as sodium laurate, potassium laurate, sodium myristate, and potassium myristate.

Phytic acid, which is employed as a metal-sequestering agent in the present invention, exhibits a metal-sequestering effect much superior to that of EDTA 4Na. In addition, since phytic acid is a natural component derived from rice bran, it causes no environmental problems. Phytic acid is available from, for example, Tsuno Rice Fine Chemicals Co., Ltd.

In the present invention, phytic acid is incorporated, in the form of being supported on starch powder, into a facial cleansing powder base. In one advantageous method for supporting phytic acid on starch powder, an aqueous phytic acid solution is kneaded with starch powder, and the thus-kneaded product is dried into a powder form. The starch employed may be cornstarch. When cornstarch is employed, the proportions between cornstarch and phytic acid are preferably 1 to 5%, more preferably 2 to 3% cornstarch for 0.1% phytic acid.

The amount of phytic acid contained in the facial cleansing powder composition of the present invention is preferably 0.01 to 2.0 wt. %, more preferably 0.05 to 0.2 wt. %. Conventionally, phytic acid has been reported to have a chelating effect comparable to that of EDTA 4Na. However, as shown in the Examples hereinbelow, the facial cleansing powder compositions, which employ phytic acid and starch in combination, exhibit latherability (in hard water) significantly superior to that of a facial cleansing powder composition containing EDTA 4Na, by virtue of a synergistic effect of phytic acid and starch.

The facial cleansing powder composition of the present invention may contain, in addition to a facial cleansing powder base and phytic acid supported on starch powder, other ingredients serving as additives which are generally incorporated into facial cleansing powder in a generally employed amounts. Examples of the additives include perfumes, enzymes (e.g., papain), and moisturizing components (e.g., placenta extract, collagen, hyaluronic acid, ceramide, and elastin).

### EXAMPLES

The present invention will next be described by way of examples, which should not be construed as limiting the invention thereto.

Facial cleansing powder compositions each containing the components shown in Table 1 in amounts (wt. %) shown therein was prepared. In Comparative Examples 1 and 2, facial cleansing powder compositions were prepared only by mixing components (in the form of powder) together. In Examples 1 and 2, an aqueous phytic acid solution was kneaded with cornstarch powder, and the thus-kneaded product was dried into a powder form. The thus-formed powder was incorporated into soap base. In the Comparative Examples and the Examples, potassium-containing soap base was employed.

In order to evaluate latherability of the facial cleansing powder compositions prepared in Comparative Examples 1 and 2 and Examples 1 and 2, each composition was dissolved in hard water (hardness: 307 mg/L) in an amount of 5 wt. %, and the aqueous solution was allowed to stand at 20° C. for 24 hours. Subsequently, the aqueous solution was placed in a 100-mL measuring cylinder, and was vigorously stirred for one minute so as to cause lathering to occur. Thereafter, the volume of lather was measured. For reference, a similar test was performed by use of purified water instead of hard water. The results are shown in Table 1.

TABLE 1

| | | Compar- | Compar- | | |
|---|---|---|---|---|---|
| | | ative | ative | Exam- | Exam- |
| | | Example 1 | Example 2 | ple 1 | ple 2 |
| Components | Soap base | 97% | 97% | 97% | 97% |
| | Cornstarch | 3% | 2.5% | 2.9% | 2.5% |
| | Phytic acid | 0 | 0 | 0.1% | 0.5% |
| | EDTA 4Na | 0 | 0.5% | 0 | 0 |
| Total | | 100% | 100% | 100% | 100% |
| Lather | Hard water | 5 mL | 4 mL | 35 mL | 38 mL |
| volume | Purified water | 40 mL | 41 mL | 40 mL | 41 mL |

As is clear from the data shown in Table 1, in the case of lathering in purified water, virtually no difference is observed in lather volume between the facial cleansing powder compositions of the Comparative Examples and the Examples. In contrast, in the case of lathering in hard water, the facial cleansing powder composition of the present invention exhibits latherability much superior to that of the facial cleansing powder composition containing EDTA 4Na.

Notably, as is clear from the data of Comparative Example 2 and Example 2, employment of phytic acid and starch in combination has a synergistic effect, although phytic acid has been conventionally reported to have a chelating effect comparable to that of EDTA.

The present invention is suitably applied to a facial cleansing powder composition to provide good latherability even in hard water, the composition containing, as a substitute for edetic acid, an environmentally acceptable metal-sequestering agent which is readily incorporated into facial cleansing powder.

What is claimed is:

1. A facial cleansing powder composition comprising a facial cleansing powder base, phytic acid, and starch powder, wherein the phytic acid is supported on the starch powder.

2. A facial cleansing powder composition as described in claim 1, which contains phytic acid in an amount of 0.01 to 2.0 wt. %.

3. A facial cleansing powder composition as described in claim 1 or 2, wherein phytic acid is supported on starch powder by mixing an aqueous phytic acid solution with starch powder and drying the mixture.
